(19) United States
(12) Patent Application Publication
(10) Pub. No.: US 2022/0228095 A1
(43) Pub. Date: Jul. 21, 2022

(54) THREE-DIMENSIONAL STRUCTURE FOR SUPPORTING CELL CULTURE MEDIUM, AND METHOD FOR MANUFACTURING FILM COMPRISING SAME

(71) Applicant: S. THEPHARM CO., LTD., Gyeonggi-do (KR)

(72) Inventor: Kyung Min Choi, Seoul (KR)

(21) Appl. No.: 17/616,632

(22) PCT Filed: May 14, 2020
(86) PCT No.: PCT/KR2020/006329

§ 371 Date: Dec. 3, 2021

(30) Foreign Application Priority Data
Jun. 5, 2019 (KR) 10-2019-0066335

(51) Int. Cl. C12M 1/12

(52) U.S. Cl. CPC C12M 25/14

(57) ABSTRACT

The present application relates to a three-dimensional structure for carrying a cell culture medium, and a method for manufacturing a film comprising same, and, more specifically, to: a three-dimensional structure for carrying a cell culture medium, comprising both a three-dimensional structure capable of carrying the proteins of a cell culture medium, and a microporous structure capable of carrying functional materials; and a method for manufacturing a film comprising same.

### TECHNICAL FIELD

The present disclosure relates to a three-dimensional structure for carrying a cell culture medium and a method of manufacturing a film including the same, and more particularly, to: a three-dimensional structure for carrying a cell culture medium, which includes both a three-dimensional structure capable of carrying the proteins or organic nutrients of a cell culture medium and a microporous structure capable of carrying functional materials: and a method of manufacturing a film including the same.

### BACKGROUND

A cell culture medium contains various proteins and organic components and has excellent biocompatibility, so it is a material that can be applied to the human body, especially the skin. However, a cell culture medium is vulnerable to denaturation by the surrounding environment and is easily contaminated with bacteria and fungi, so it is difficult to distribute or use it as a product. In order to deliver a cell culture medium having these characteristics to the skin, various techniques have been proposed to date.

Among the techniques, there is a method of delivering a cell culture medium to the skin after carrying it in a liposome-type capsule. However, since liposomes are based on weak bonds, they are easily denatured or broken, so there are limitations to using these structures to deliver a cell culture medium.

In addition, since the technique takes the approach of protecting only a single component or injecting an antimicrobial material, it is not possible to protect all components in a cell culture medium.

In addition, conventionally, when manufacturing a mask pack using a cell culture medium, a structure for carrying the cell culture medium is mostly formed at high temperature by using thermal energy. However, this is a process that cannot be used for a cell culture medium, which is sensitive to heat.

Therefore, it is necessary to study a structure for carrying a cell culture medium that is capable of delivering various components of the cell culture medium to the skin as intended by the user and is resistant to high temperature.

### RELATED-ART DOCUMENT

(Patent Document 1) Korean Patent Publication No. 10-2015-0004750 (published on Jan. 13, 2015)

### DISCLOSURE

### Technical Problem

The present disclosure is directed to providing a three-dimensional structure capable of carrying various materials, including a cell culture medium, and a method of manufacturing a hydrogel film at low temperature.

### Technical Solution

One aspect of the present disclosure provides a method of manufacturing a film, which includes: mixing a cell culture medium, metal-organic frameworks (MOFs), poly(ethylene glycol) diacrylate (PEGDA), a photocuring agent, and glycerol to form a mixture; applying the mixture in a predetermined shape; and irradiating the applied mixture with light to form a gelled film.

Another aspect of the present disclosure provides a three-dimensional structure including a cell culture medium including proteins and organic nutrients, which includes: a PEGDA polymer network having a three-dimensional network structure; MOFs dispersed within the PEGDA polymer network and including a plurality of pores; proteins dispersed within the PEGDA polymer network and/or located in the plurality of pores of the MOFs; and organic nutrients located in the plurality of pores of the MOFs.

Still another aspect of the present disclosure provides a hydrogel film including the above-described three-dimensional structure.

Yet another aspect of the present disclosure provides a skin article formed of the above-described hydrogel film, wherein the article is a mask pack or a mask patch.

### Advantageous Effects

According to one embodiment of the present disclosure, it is possible to provide a three-dimensional structure having pores or structural characteristics capable of carrying a cell culture medium including materials of various sizes.

According to one embodiment of the present disclosure, it is possible to provide a three-dimensional structure capable of securely fixing a material to be supported using a strong bond.

According to one embodiment of the present disclosure, it is possible to provide a three-dimensional structure capable of preventing the denaturation of a cell culture medium by bacteria or fungi.

According to one embodiment of the present disclosure, it is possible to provide a hydrogel film at low temperature or room temperature, without denaturing a cell culture medium.

According to one embodiment of the present disclosure, it is possible to provide a soft or hard hydrogel film by controlling the properties of a film composition.

According to one embodiment of the present disclosure, it is possible to provide a mask pack or a mask patch using the above-described film.

### DESCRIPTION OF DRAWINGS

FIG. 1 is a flowchart illustrating a method of manufacturing a hydrogel film according to one embodiment of the present disclosure.

FIG. 2 shows an image of a cell culture medium according to one embodiment of the present disclosure and a schematic diagram for illustrating components thereof.

FIG. 3 shows images for illustrating a three-dimensional structure according to one embodiment of the present disclosure.

FIG. 4 shows images for illustrating a three-dimensional structure according to one embodiment of the present disclosure.

FIG. 5 is an image of a film-forming composition in which a cell culture medium according to one embodiment of the present disclosure is carried.

FIG. 6 shows images of mask patches manufactured using a film-forming composition in which a cell culture medium according to one embodiment of the present disclosure is carried.

FIG. 7 shows images of film-forming compositions including a cell culture medium and metal organic frameworks (MOFs) by concentration according to one embodiment of the present disclosure.

FIG. 8 shows images of mask patches manufactured using film-forming compositions including a cell culture medium and metal organic frameworks (MOFs) by concentration according to one embodiment of the present disclosure.

FIG. 9 shows images of mask patches according to one embodiment of the present disclosure immediately after manufacture using film-forming compositions according to glycerol content.

FIG. 10 shows images of mask patches according to one embodiment of the present disclosure three hours after manufacture using film-forming compositions according to glycerol content.

FIG. 11 is an image of a mask patch manufactured using a nonwoven fabric according to one embodiment of the present disclosure.

### BEST MODE

Terms used in the present disclosure are only used to describe exemplary embodiments and are not intended to limit the present disclosure. Singular expressions include plural expressions unless the context clearly dictates otherwise. In the present disclosure, terms such as “comprise,” “include,” “contain,” or “have” are intended to indicate the presence of features, components, elements, or the like described herein, and do not preclude the possibility of the presence or addition of one or more other features, components, elements, or the like.

Unless defined otherwise, all terms used herein, including technical or scientific terms, have the same meaning as commonly understood by one of ordinary skill in the art to which the present disclosure pertains. Terms such as those defined in commonly used dictionaries should be interpreted as having a meaning consistent with the meaning in the context of the related art and should not be interpreted in an ideal or excessively formal sense unless explicitly defined in the present disclosure.

In the present disclosure, the term “nano” may mean a size of nanometers (nm), and for example, may mean a size of 1 nm to 1,000 nm, but the present disclosure is not limited thereto. In addition, the term “nanoparticle” used herein may mean a particle having an average particle diameter of nanometers (nm), and for example, may mean a particle having an average particle diameter of 1 nm to 1,000 nm, but the present disclosure is not limited thereto.

Hereinafter, a method of manufacturing a hydrogel film of the present disclosure will be described in detail with reference to the accompanying drawings. However, the accompanying drawings are exemplary, and the scope of a method of manufacturing a hydrogel film of the present disclosure is not limited by the accompanying drawings.

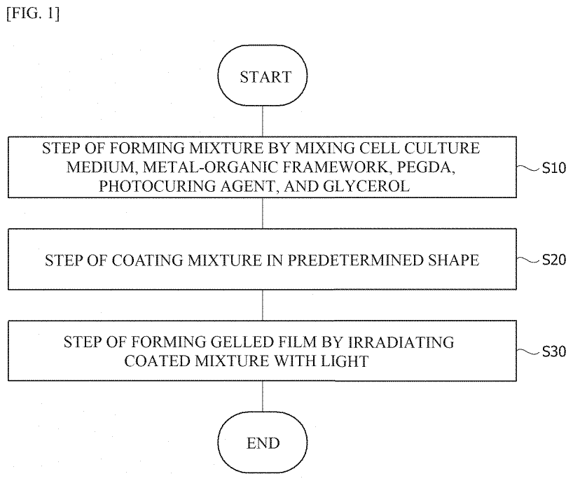

FIG. 1 is a flowchart illustrating a method of manufacturing a hydrogel film according to one embodiment of the present disclosure.

As illustrated in FIG. 1, the method of manufacturing a hydrogel film according to one embodiment of the present disclosure includes: mixing a cell culture medium, metal organic frameworks (MOFs), PEGDA, a photocuring agent, and glycerol to form a mixture (S10); applying the mixture in a predetermined shape (S20); and irradiating the applied mixture with light to form a gelled film (S30).

Here, the mixing ratio of the mixture is 1 to 50 parts by weight of the MOFs, 3 to 10 parts by weight of the PEDGA, 0.01 to 0.1 parts by weight of the photocuring agent, and 0 to 40 parts by weight of the glycerol based on 100 parts by weight of the cell culture medium.

Hereinafter, steps of the present disclosure will be described in more detail.

First, a cell culture medium, MOFs, PEGDA, a photocuring agent, and glycerol are mixed to form a mixture (S10).

Although there is no particular limitation on the order of adding the components, it is preferable that the components are added in the following order i) cell culture medium, ii) PEGDA, iii) MOFs, and iv) photocuring agent.

The term “cell culture medium” used herein refers to a solution including organic nutrients and proteins discharged in the process of cell growth. The present disclosure does not impose particular limitations on the types of proteins or organic nutrients included in the cell culture medium, and relates to a technique of carrying the proteins or organic nutrients. In addition, there is no particular limitation on a method of obtaining the proteins or organic nutrients, and they can be obtained by a known method.

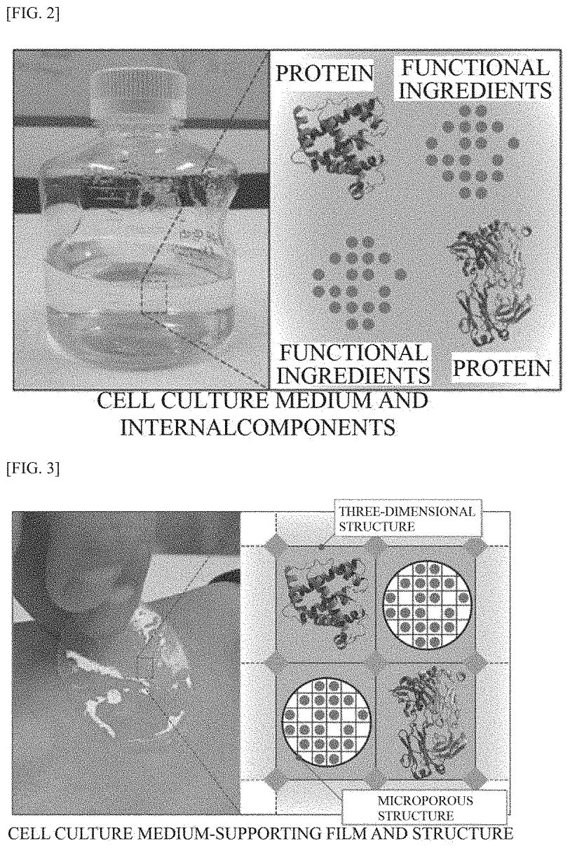

FIG. 2 shows an image of a cell culture medium according to one embodiment of the present disclosure and a schematic diagram for illustrating components thereof.

As shown in FIG. 2, the cell culture medium includes various proteins, organic components, and water.

In addition, the cell culture medium may be further diluted with water and the weight proportion of the proteins and organic nutrients in the cell culture medium may be 5 wt % to 95 wt %.

The weight proportion of the above-described cell culture medium may vary, and when concentration is high, the cell culture medium may be diluted with water and used according to the intention of the present disclosure or an intended use.

Poly(ethylene glycol) (PEG) is a type of polymer that forms a network structure, and functions as a support for forming a hydrogel film by forming a network structure.

In the case of PEGDA, when used together with a photocuring accelerator, the diacrylate attached to two ends of PEG is bonded with each other upon exposure to ultraviolet (UV) wavelengths and forms a network structure, which can act as a support capable of carrying and carrying MOFs and a cell culture medium. The PEGDA is preferably included in an amount of 3 parts by weight to 10 parts by weight based on 100 parts by weight of the cell culture medium. When the content is less than 3 parts by weight, a support having a network structure intended in the present disclosure cannot be formed. On the other hand, when the content exceeds 10 parts by weight, excessive curing occurs during UV curing, so a cell culture medium cannot be effectively supported therein.

The glycerol is used for preventing moisture evaporation and ensuring the fluidity of a produced film. When a PEGDA-based film is formed without glycerol, an excessively cured film may be formed, or sufficient strength for use in a product is not secured, and thus it tends to be easily crushed. In addition, the glycerol inhibits the rapid evaporation of moisture so that water is well retained in the cell culture medium, maintains the strength of a PEGDA film, and secures fluidity.

Here, the glycerol is preferably included in an amount of 10 parts by weight to 30 parts by weight based on 100 parts by weight of the cell culture medium. However, the glycerol content has the greatest influence on the properties of a film. When the content is less than 10 parts by weight, a film with high hardness is formed. When the content is in the range of 10 parts by weight to 30 parts by weight, since a sufficient amount of cell culture medium is contained, a film having excellent adhesion with the skin and a low degree of moisture evaporation can be formed. In addition, since mechanical strength is also excellent, when used alone or in combination with a nonwoven fabric, a film that can be attached to/detached from the skin and folded/unfolded without difficulty can be provided. In addition, water contained in the cell culture medium can be maintained in the film for one or more days. On the other hand, when the amount exceeds 30 parts by weight, due to the high glycerol content, the viscosity of a film-forming composition becomes excessively high, and the strength of a film formed after a photocuring reaction is significantly reduced.

The photocuring agent promotes the bonding of diacrylate by absorbing light and forms a network structure in a PEG support. Although various photocuring agents known in the art to which the present disclosure pertains may be used, 2-hydroxy-4′-(2-hydroxyethoxy)-2-methylpropiophenone (Irgacure 2959) is most preferable under conditions where a cell culture medium, MOFs, and glycerol are present. The photocuring agent is preferably included in an amount of 0.01 parts by weight to 1 part by weight based on 100 parts by weight of the cell culture medium.

The MOFs are porous materials in which an organic linker (or an organic bridging ligand) is connected to a metal oxide by a coordination bond, thus forming a three-dimensional structure. Since the MOFs basically have a very large surface area and an open pore structure, they are capable of carrying a larger amount of molecules, solvents, and the like than other conventionally known porous materials or allowing the same to move therethrough, and it is possible to make changes to the resulting central metal-organic ligand framework or components thereof and control the size (volume) of pores.

Hereinafter, the skeletal structure of the MOF will be described in more detail. The MOFs include metal clusters, and the clusters are linked together in a periodic manner by organic linkers (linking ligands, organic bridging ligands) that increase the distance between the clusters to provide a network-like structure. For example, the organic linkers refer to chemical species that coordinate to two or more metals (including neutral molecules and ions), and since the organic linkers form gaps between metals, empty spaces, i.e., pores, may be formed in a resulting structure. Examples of the organic linker include 4,4′-bipyridine (neutral, multiple N-donor molecule) and benzene-1,4-dicarboxylate (polycarboxylate anion).

Metal ions used in the MOFs include one or more ions selected from the group consisting of metals of Groups 1 to 16 (including actinides and lanthanides) of the IUPAC Periodic Table of Elements. Specific examples of the metal ions used in the MOFs of the present disclosure include one or more ions selected from the group consisting of the following ions: Li⁺, Na⁺, K⁺, Rb⁺, Be²⁺, Mg²⁺, Ca²⁺, Sr²⁺, Ba²⁺, Sc³⁺, Y³⁺, Ti⁴⁺, Zr⁴⁺, Hf⁴⁺, V⁴⁺, V³⁺, V²⁺, NB³⁺, Ta³⁺, Cr³⁺, Mo³⁺, W³⁺, Mn³⁺, Mn²⁺, Re³⁺, Re²⁺, Fe³⁺, Fe²⁺, Ru³⁺, Ru²⁺, Os³⁺, Os²⁺, Co³⁺, Co²⁺, Rh²⁺, Rh⁺, Ir²⁺, Ir⁺, Ni²⁺, Ni⁺, Pd²⁺, Pd⁺, Pt²⁺, Pt⁺, Cu²⁺, Cu⁺, Ag⁺, Au⁺, Zn²⁺, Cd²⁺, Hg²⁺, Al³⁺, Ga³⁺, In³⁺, Tl³⁺, Si⁴⁺, Si²⁺, Ge⁴⁺, Ge²⁺, Sn⁴⁺, Sn²⁺, Pb⁴⁺, Pb²⁺, As⁵⁺, As³⁺, As⁺, Sb⁵⁺, Sb³⁺, Sb⁺, Bi⁵⁺, Bi³⁺, Bi⁺, and combinations thereof. Among these ions, Co²⁺, Cu²⁺, Zn²⁺, and Zr⁴⁺ are preferable because of their ability to form predetermined clusters in a synthetic mixture.

The organic linkers may be charged organic linkers. The charged organic linkers include anionic functional groups such as carboxylate (CO₂—), sulfate (SO₃—), and the like. In addition, in general, each organic linker may include two or more charged functional groups. The organic linkers may be bidentate ligands or tridentate ligands (a larger number of, i.e., more than three, functional groups are also within the scope of the present disclosure). Therefore, examples of useful organic linkers may include two or more carboxylate groups.

As described above, since the organic linker forms a gap between metal clusters, the size of pores can be controlled by the length of the organic linker.

Here, the MOFs may be included in an amount of 1 part by weight or more based on 100 parts by weight of the cell culture medium, but since a produced film tends to be hard and brittle as MOF content increases, the MOFs are preferably included in an amount of 1 part by weight to 50 parts by weight.

The average particle size of the MOFs is preferably 1 nm to 100 μm. When this range is satisfied, it is possible to form an emulsion well dispersed in the cell culture medium, and it is possible to support and protect 1 to 100 parts by weight of a functional material or active ingredient based on 100 parts by weight of a mixture of molecular-level materials such as antioxidants, vitamins, and anti-aging agents.

As described above, the MOFs have pores whose size can be adjusted according to a structure formed by the bonding between linkers and metals, and each of the pores has a size of 9.0 to 20 Å and thus is capable of effectively protecting molecular-level functional materials. In addition, when the MOFs are designed to have large pores (2 nm to 1,000 nm) for containing and protecting proteins, previously formed micropores may be partially removed to form large pores, or MOFs having large pores may be formed using a macropore-inducing agent in the MOF manufacturing step.

As described above, the mixture may include a functional material. The functional material includes at least one selected from the group consisting of an antioxidant, a vitamin, and an anti-aging agent. In this case, the functional material may be used as a starting material in a state included in the MOF.

In addition, the mixture prepared as described above may be homogenized. There is no particular limitation on a method of homogenizing the mixture, and any method may be used as long as it is applicable in the art to which the present disclosurepertains. Stirring may be performed for 2 to 60 minutes using a stirrer, or sonication may be performed for one minute or less.

Next, the mixture is applied in a predetermined shape (S20).

Here, the predetermined shape may be the shape of a final film determined in advance in consideration of the user's intention, and the mixture may be applied in that shape. The shape may be a shape of a mask pack or a shape of a mask patch.

In this case, the mixture may be applied onto a nonwoven fabric. When a nonwoven fabric is used, the final film can be more securely supported. Any substrate capable of exhibiting a function similar to that of a nonwoven fabric may be used. Although there is no particular limitation on the shape of the nonwoven fabric, a nonwoven fabric formed in the shape of a final film determined in advance in consideration of the users intention may be used, or the mixture may be applied onto a nonwoven fabric without a particular shape and then cut into a desired shape at a later time.

Subsequently, the applied mixture is irradiated with light, and thus a gelled film is formed (S30).

When light is applied, the gelation of PEGDA can be accelerated, and a hydrogel film can be formed. Through this, the above-described shape of the three-dimensional structure can be more securely fixed.

The light is preferably UV light. Although there is no particular limitation on the light source, it is preferable to use a light-emitting diode (LED) light source. There is no particular limitation on the wavelength of the LED light, and the wavelength may be 450 to 490 nm or 460 to 480 nm, preferably 470 nm.

Here, the light irradiation is preferably performed for 5 to 60 seconds using an LED light source. When the light is applied for less than 5 seconds, sufficient gelation does not occur, and when the light is applied for more than 60 seconds, the increase in effect is insignificant.

In addition, when using a nonwoven fabric as described above, it is possible to provide a more robust film because the nonwoven fabric is used as a support for the hydrogel film.

FIG. 3 shows a three-dimensional structure according to one embodiment of the present disclosure. As shown in FIG. 3, in a prepared mixture, a three-dimensional structure including a PEGDA polymer network having a three-dimensional structure, MOFs dispersed within the PEGDA polymer network and including a plurality of pores, proteins dispersed within the PEGDA polymer network and/or located in the plurality of pores of the MOFs, and organic nutrients located in the plurality of pores of the MOFs may be formed.

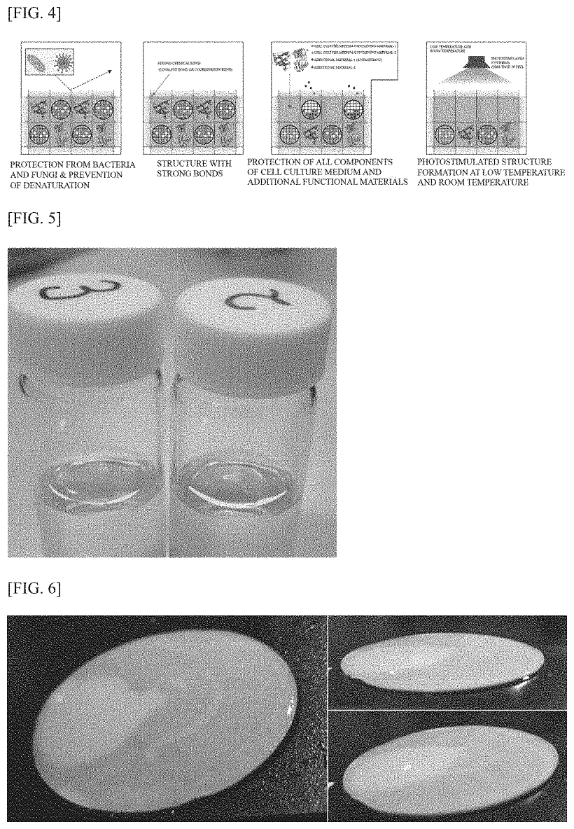

In addition, FIG. 4 shows a three-dimensional structure according to one embodiment of the present disclosure. As shown in FIG. 4, a film including a three-dimensional structure manufactured by the above-described method protects a cell culture medium from bacteria or fungi and securely supports the structure using strong chemical bonds, and is capable of carrying all components of a cell culture medium and optionally an additional functional material and forming a structure at low temperature and room temperature by photostimulation.

Another aspect of the present disclosure provides a three-dimensional structure. The three-dimensional structure includes: a PEGDA polymer network having a three-dimensional network structure; MOFs dispersed within the PEGDA polymer network and including a plurality of pores; proteins dispersed within the PEGDA polymer network and/or located in the plurality of pores of the MOFs; and organic nutrients located in the plurality of pores of the MOFs.

Descriptions of the same or similar components as those described in the above-described method of manufacturing a film will be omitted.

The PEGDA polymer network has a three-dimensional network structure and serves as a support for another component to be described below. In the network structure, MOFs are dispersedly provided.

Since the MOFs have a plurality of pores, organic nutrients with a small size provided from a cell culture medium are supported in the pores, and MOFs controlled to have a large pore size are also capable of carrying proteins with a relatively large size. In addition, a functional material that is additionally included can also be supported. In general, other proteins with a large size can generally be present within a PEGDA polymer network structure.

Preferably, the MOFs have an average particle size of 1 nm to 100 μm, and the average diameter of the plurality of pores is in the range of 9.0 Å to 20 Å.

Such structural hierarchy allows materials of various sizes to be securely supported.

In particular, when the pore size is large, proteins may also be located in the pores and thereby included within the MOFs.

Preferably, the three-dimensional structure is controlled to have a component ratio of 1 to 20 parts by weight of the MOFs, 3 to 10 parts by weight of PEDGA, 0.01 to 0.1 parts by weight of the photocuring agent, and 0 to 40 parts by weight of the glycerol based on 100 parts by weight of the proteins and organic nutrients.

Here, the proteins and organic nutrients are preferably derived from a cell culture medium.

In addition, the three-dimensional structure may additionally include a functional material located in the plurality of pores. In addition, the functional material includes at least one selected from the group consisting of an antioxidant, a vitamin, and an anti-aging agent.

Still another aspect of the present disclosure provides a hydrogel film including the above-described three-dimensional structure. Although there is no limitation, the hydrogel film may be manufactured by the above-described method of manufacturing a film.

The hydrogel film is a film manufactured by UV irradiation, contains an appropriate amount of moisture, and has appropriate strength. In order to increase strength, a nonwoven fabric may be attached to one side.

Yet another aspect of the present disclosure provides a skin article formed of the above-described hydrogel film, wherein the article is a mask pack or a mask patch.

Through the mask pack or mask patch, it is possible to provide a skin care product capable of effectively protecting various components of a cell culture medium using the above-described three-dimensional structure and delivering the cell culture medium components to the skin. In addition, it is possible to perform control so that the above-described structure is formed at low temperature or room temperature, so that components of the cell culture medium are not destroyed or denatured.

### MODES OF THE INVENTION

Hereinafter, the present disclosure will be described in more detail through Experimental Examples.

### Experimental Example 1

10 g of a cell culture medium, 1 g of PEGDA, 2, 3, or 4 g of glycerol, and 0.01 g of a photocuring agent were mixed. After subsequently adding 0.5 g of MOFs, the mixture was stirred for 20 minutes using a stirrer.

FIG. 5 is an image of the obtained film-forming composition carrying a cell culture medium. In FIG. 5, a solution containing 3 g of glycerol is shown on the left side, and a solution containing 2 g of glycerol is shown on the right side. Through this, it was possible to determine the optimal glycerol content.

The above-prepared cell culture medium was applied and irradiated with an UV LED lamp for 20 seconds, and thus patches were produced.

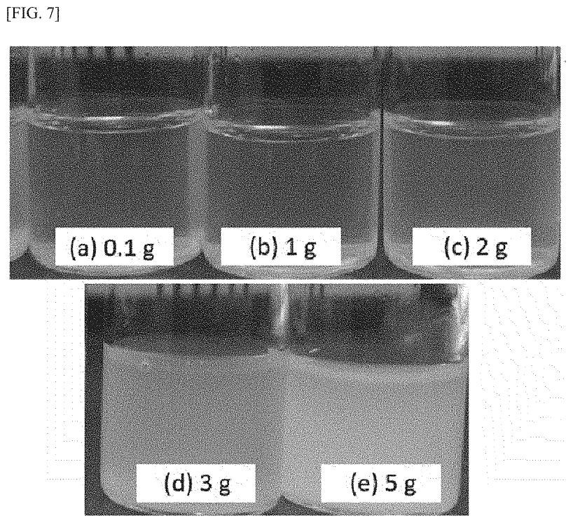

FIG. 6 shows images of the produced mask patches. As shown in FIG. 6, it can be seen that films were successfully formed even under conditions where 2, 3, or 4 g of glycerol was present.

### Experimental Example 2

An additional experiment was conducted to study the difference in compositions and films according to MOF content.

10 g of a cell culture medium, 2 g of PEGDA, 2 or 3 g of glycerol, and 0.01 g of a photocuring agent were mixed. After subsequently adding 0.1, 1, 2, 3, or 5 g of MOFs, the mixture was stirred for 30 minutes using a stirrer, and thus five types of compositions were prepared.

FIG. 7 shows images of film-forming compositions according to MOF content. Referring to FIG. 7, it can be seen that the MOFs are uniformly and effectively dispersed in the compositions under all conditions.

In addition, among the above-described five types of compositions, compositions in which MOF content was controlled to 1, 2, or 5 g were applied in a circular shape and irradiated with an UV LED for 30 seconds, and thus hydrogel films were produced.

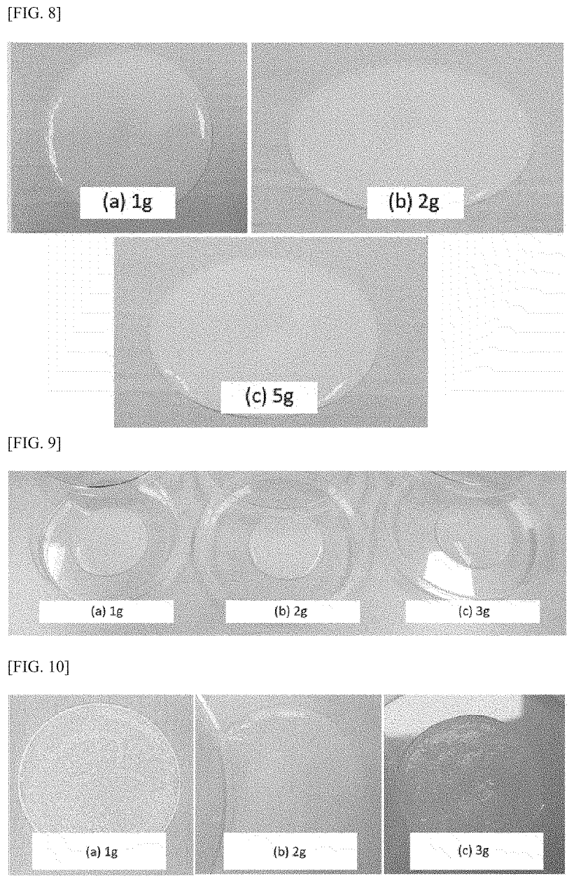

FIG. 8 shows images of the produced mask patches. As shown in FIG. 8, all three types were formed as robust hydrogel films.

### Experimental Example 3

An additional experiment was conducted to study the difference in films according to glycerol content.

10 g of a cell culture medium, 0.5 g of MOFs, 1 g of PEGDA, 1, 2, or 3 g of glycerol, and 0.01 g of a photocuring agent were mixed and stirred for 20 minutes using a stirrer.

Three types of compositions were applied in a circular shape and irradiated with an UV LED for 30 seconds, and thus their respective films were produced.

FIG. 9 shows images of mask patches immediately after manufacture using film-forming compositions according to glycerol content. In addition, FIG. 10 shows images of mask patches three hours after manufacture using film-forming composition according to glycerol content.

Through this, it can be seen that the lower the glycerol content, the faster the film dries and the higher the hardness, and the higher the glycerol content, the slower the film dries but the lower the hardness is. It was confirmed that there was an optimal glycerol concentration according to compositions used.

### Experimental Example 4

An additional experiment was conducted to study the difference in films according to the presence of a nonwoven fabric capable of carrying a film.

10 g of a cell culture medium, 0.5 g of MOFs, 1 g of PEGDA, 1, 2, or 3 g of glycerol, and 0.01 g of a photocuring agent were mixed and stirred for 20 minutes using a stirrer, and thus a composition was prepared.

The composition was applied onto a prepared circular porous nonwoven fabric and irradiated with an UV LED for 30 seconds, and thus a hydrogel film was produced.

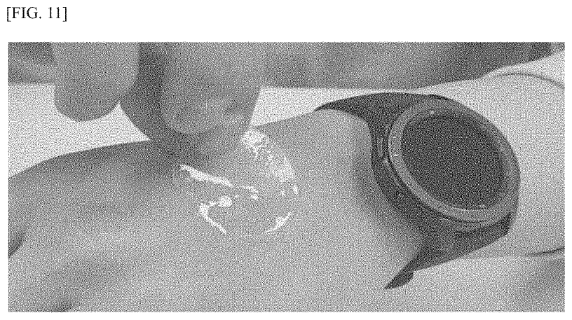

FIG. 11 is an image of a mask patch manufactured using a nonwoven fabric. Referring to FIG. 11, it can be seen that a more securely supported hydrogel patch has been formed.

Although the present disclosure has been described above with reference to exemplary embodiments, it will be understood by those skilled in the art that various modifications and changes can be made to the present disclosure within the scope not departing from the spirit and scope of the present disclosure as set forth in the appended claims.

1. A method of manufacturing a film, comprising:
mixing a cell culture medium, metal-organic frameworks, poly(ethylene glycol) diacrylate (PEGDA), a photocuring agent, and glycerol to form a mixture;
applying the mixture in a predetermined shape; and
irradiating the applied mixture with light to form a gelled film.

2. The method of claim 1, wherein the step of applying the mixture in a predetermined shape is a step of applying the mixture in the predetermined shape onto a prepared nonwoven fabric.

3. The method of claim 1, wherein the cell culture medium includes proteins and organic nutrients and the weight proportion of the proteins and the organic nutrients in the cell culture medium is 5 wt % to 95 wt %.

4. The method of claim 1, wherein a mixing ratio of the mixture is 1 part by weight to 50 parts by weight of the metal-organic frameworks, 3 parts by weight to 10 parts by weight of the PEDGA, 0.01 parts by weight to 0.1 parts by weight of the photocuring agent, and 0 parts by weight to 40 parts by weight of the glycerol based on 100 parts by weight of the cell culture medium.

5. The method of claim 1, wherein the mixture further includes 1 part by weight to 100 parts by weight of a functional material based on 100 parts by weight of the mixture.

6. The method of claim 5, wherein the functional material includes at least one selected from the group consisting of an antioxidant, a vitamin, and an anti-aging agent.

7. The method of claim 1, wherein the step of forming a mixture includes stirring the mixture for 1 minute to 60 minutes or sonicating the mixture for one minute or less.

8. The method of claim 1, wherein the irradiation with light includes irradiation with a light-emitting diode (LED) source light for 5 seconds to 60 seconds.

9. The method of claim 1, wherein the predetermined shape is a predetermined shape of a mask pack or mask patch.

10. A three-dimensional structure comprising a cell culture medium including proteins and organic nutrients, the structure comprising:
a PEGDA polymer network having a three-dimensional network structure;
metal-organic frameworks dispersed within the PEGDA polymer network and including a plurality of pores;
proteins dispersed within the PEGDA polymer network and/or located in the plurality of pores of the metal-organic frameworks; and
organic nutrients located in the plurality of pores of the metal-organic frameworks.

11. The three-dimensional structure of claim 10, further comprising glycerol and/or a photocuring agent dispersed within the PEGDA polymer network.

12. The three-dimensional structure of claim 11, having a component ratio of 1 part by weight to 50 parts by weight of the metal-organic frameworks, 3 parts by weight to 10 parts by weight of the PEDGA, 0.01 parts by weight to 0.1 parts by weight of the photocuring agent, and 0 parts by weight to 40 parts by weight of the glycerol based on 100 parts by weight of the cell culture medium.

13. The three-dimensional structure of claim 10, wherein an average particle size of the metal-organic frameworks is in the range of 1 nm to 100 μm.

14. The three-dimensional structure of claim 10, wherein an average diameter of the plurality of pores is in the range of 9.0 Å to 20 Å.

15. The three-dimensional structure of claim 10, further comprising a functional material located in the plurality of pores.

16. The three-dimensional structure of claim 15, wherein the functional material includes at least one selected from the group consisting of an antioxidant, a vitamin, and an anti-aging agent.

17. A hydrogel film comprising the three-dimensional structure of claim 10.

18. The hydrogel film of claim 17, wherein a nonwoven fabric is attached to one side of the film.

19. A skin article formed of the hydrogel film of claim 18, wherein the article is a mask pack or a mask patch.
